(19) United States
(12) Patent Application Publication
(10) Pub. No.: US 2024/0050413 A1
(43) Pub. Date: Feb. 15, 2024

(54) COMPOSITIONS AND METHODS FOR TREATING MULTIPLE SCLEROSIS AND ALLEVIATING FATIGUE

(71) Applicant: University of Miami, Miami, FL (US)

(72) Inventor: Kottil W. Rammohan, Miami, FL (US)

(21) Appl. No.: 18/028,338

(22) PCT Filed: Sep. 24, 2021
(86) PCT No.: PCT/US2021/051930

§ 371 Date: Mar. 24, 2023

Related U.S. Application Data
(60) Provisional application No. 63/082,975, filed on Sep. 24, 2020.

(51) Int. Cl. A61K 31/4172; A61K 31/195; A61P 21/00

(52) U.S. Cl. CPC A61K 31/4172; A61K 31/195; A61P 21/00

(57) ABSTRACT

Disclosed herein are compositions and methods for treating multiple sclerosis and alleviating fatigue.

### CROSS-REFERENCE TO RELATED APPLICATIONS

This application claims the benefit of U.S. Provisional Application No. 63/082,975, filed Sep. 24, 2020, which is expressly incorporated herein by reference in its entirety.

### STATEMENT REGARDING FEDERALLY SPONSORED RESEARCH

This invention was made with government support under Grant No. W81XWH-16-1-0462 awarded by the Department of Defense. The Government has certain rights in the invention.

### FIELD

This disclosure relates generally to methods for increasing brain histamine levels in multiple sclerosis patients. The disclosure provides methods for treating multiple sclerosis. The disclosure also provides for a sequential dosing strategy to alleviate fatigue.

### BACKGROUND

The mechanism of fatigue in multiple sclerosis (MS) remains elusive. Fatigue is experienced by over 75% of MS patients with MS at some time during the course of their disorder. The severely disabling fatigue is identified as a major contributor to unemployment in a large number of patients with MS. Further, this symptom interferes with and worsens other symptom of MS especially depression, cognition, pain and motor function amongst others.

Treatment of fatigue may alleviate these other symptoms of MS to the extent that the symptoms are worsened by fatigue, but each of these symptoms have their own mechanisms of pathogenesis. Accordingly, while patients may report improvement of cognition when their fatigue is successfully treated, cognitive dysfunction in MS cannot be treated by simply alleviating fatigue. Nevertheless, alleviating fatigue can have a significant impact on the overallquality of life of the individual.

Current agents that alleviate fatigue in MS are generally stimulants, which act through enhancement of the function of the noradrenergic brain. Stimulants of the phenethylamine class, dominated by the amphetamine salts, both levo and dextro isomers, used for treatment of attention deficit and sleep disorders have also found use in treatment of fatigue in MS. However, the effectiveness of these agents limits their use in treating the fatigue associated with MS. Integrally associated with the suprachiasmatic nucleus, the master clock of the body, the tuberomammillary system is responsible for control of the circadian rhythm, wakefulness and sleep, satiety and hunger, libido and sexual arousal, and learning and memory. The sole neurotransmitter of these neurons is histamine. However, histamine itself is unable to cross the blood-brain barrier and act centrally.

### SUMMARY

Disclosed herein are methods for increasing brain histamine by administering the precursor L-histidine which retains the ability to access the central nervous system (CNS) by way of amino acid transporters. Also disclosed herein are methods that overcome the limitations of peripherally administered histamine.

In some aspects, disclosed herein is a method for treating multiple sclerosis in a subject in need thereof, comprising administering to the subject a therapeutically effective amount of L-histidine.

In some embodiments, the method of any preceding aspect further comprises administering to the subject a therapeutically effective amount of carbidopa. In some embodiments, carbidopa and L-histidine are taken daily. In some embodiments, the therapeutically effective amount of carbidopa is unchanged and the therapeutically effective amount of L-histidine is increased over a three week period. In some embodiments, the dose or therapeutically effective amount of carbidopais from about 25 mg per day to about 75 mg per day. In some embodiments, the dose or therapeutically effective amount of carbidopa is about 50 mg per day. In some embodiments, the dose or therapeutically effective amount of L-histidineis from about 250 mg per day to about 2000 mg per day. In some embodiments, the dose or therapeutically effective amount of L-histidineis from about 250 mg per day to about 1000 mg per day. In some embodiments, L-histidine is administered orally, intravenously, or intramuscularly.

In some embodiments, carbidopa is administered concurrently, before, or after L-histidine. In some embodiments, carbidopa and L-histidine are administered orally.

In some embodiments, the method of any preceding aspect alleviates fatigue in the subject.

In some aspects, disclosed herein is a method for alleviating fatigue in a subject in need thereof, the method comprising administering a therapeutically effective amount of L-histidine. In some embodiments, the subject has multiple sclerosis.

In some embodiments, the method further comprises administering to the subject a therapeutically effective amount of carbidopa. In some embodiments, carbidopa and L-histidine are taken daily and the dose of carbidopa is unchanged and the dose of L-histidine is increased over a three week period. In some embodiments, the dose of carbidopa is from about 25 mg per day to about 75 mg per day, or more specifically, about 50 mg per day. In some embodiments, the doses of L-histidine are from about 250 mg per day to about 2000 mg per day, more specifically about 250 mg per day to about 1000 mg per day.

In some embodiments, the carbidopa is administered concurrently, before, or after the L-histidine and both are administered orally. In some embodiments, the L-histidine is administered orally, intravenous (IV), or intramuscular (IM).

In some aspects, disclosed herein is a method for alleviating fatigue in individuals with multiple sclerosis, the method comprising oral administration of L-histidine.

In some embodiments, the method of any preceding aspect increases a level of N-acetylaspartate in the central nervous system of the subject. In some embodiments, the method of any preceding aspects increases a level of creatine phosphate in the central nervous system of the subject.

In some aspects, disclosed herein is a method for treating a neurological disorder in a subject in need, comprising administering to the subject a therapeutically effective amount of L-histidine and/or carbidopa. In some embodiments, the neurological disorder is Alzheimer's Disease, Parkinson's disease, or Amyotrophic Lateral Sclerosis (ALS)

These and other features and advantages of the present invention will be more fully understood from the following detailed description taken together with the accompanying drawings and claims. It is noted that the scope of the claims is defined by the recitations therein and not by the specific discussion of features and advantages set forth in the present description.

### BRIEF DESCRIPTION OF THE DRAWINGS

The accompanying figures, which are incorporated in and constitute a part of this specification, illustrate several aspects described below.

FIG. 1 shows a design for studying histaminergic basis of fatigue in multiple sclerosis.

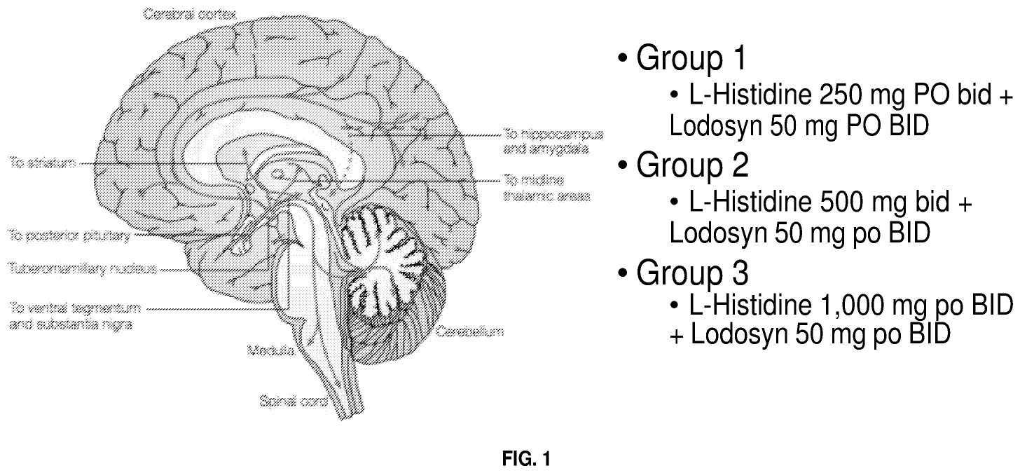

FIG. 2 shows Modified Fatigue Index Scale (MFIS) changes between screening visit (1),baseline or off drug (2), and at day 8 or on drug; of the study regimen (3). Subjects were administered 250 mg daily of L-Histidine.

FIG. 3 shows Modified Fatigue Index Scale (MFIS) changes between screening visit (1), baseline or off drug (2), and at day 8 or on drug; of the study regimen (3). Subjects were administered 500 mg daily of L-Histidine.

FIG. 4 shows Modified Fatigue Index Scale (MFIS) changes between screening visit (1),baseline or off drug (2), and at day 8 or on drug; of the study regimen (3). Subjects were administered 1000 mg daily of L-Histidine.

FIGS. 5A-5B show pharmacokinetic data for L-Histidine in plasma (FIG. 5A) and CSF (FIG. 5B) after the 500 mg carbidopa arm.

FIG. 6 shows the data of heat map analysis. Dark red and dark green cells represent statistically significant (p≤0.05) increases or decreases, respectively. Light red and light green cells are trending (0.05<p<0.10) higher or lower, respectively.

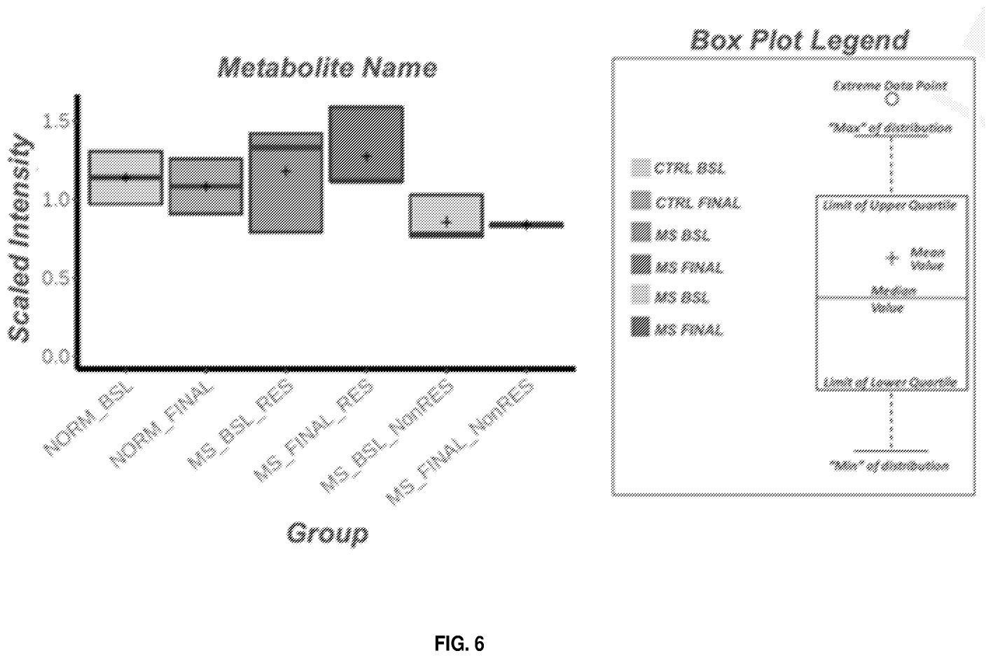

FIG. 7 shows data of histidine metabolism. Histamine levels in the CSF were below the level of detection sensitivity. Histidine levels were normal in MS and normal. Histidine levels increased after loading similarly in MS patients and normal subjects. N-acetylcarnosine which is acetylated in the mitochondria were trending lower in MS patients than normal subjects, and with homocarnosine indicate the redox state of the cell. Carnosine and homocarnosine are histidine containing dipeptides that have neuroprotective properties. Their levels were not abnormal and did not change significantly after histidine loading.

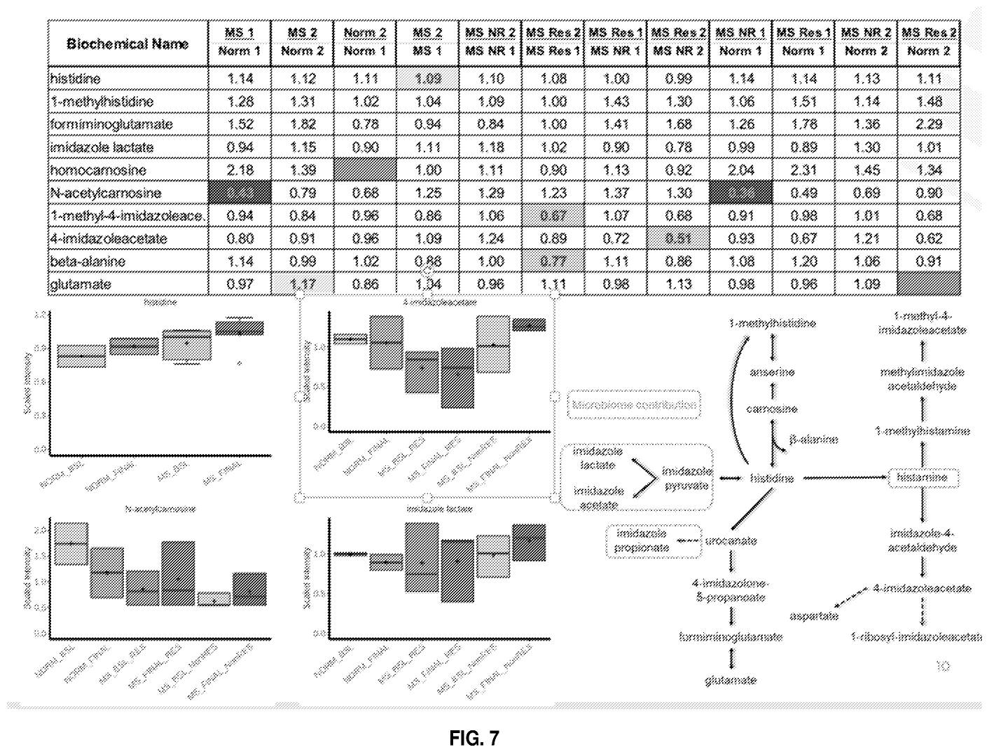

FIG. 8 shows metabolites from mitochondrial activity. N-acetylaspartate (NAA) is exclusively a marker of neuronal health and synthesized in the neuronal mitochondria. It is increased in the responder population after histidine loading. Creatine phosphate (ATP) is generated in the mitochondria by oxidation of glucose or fatty acids. It is increased in the responder population after histidine loading.

FIG. 9 show data of fatty acid metabolism. Fatty acid beta oxidation is increased with generation of ketone bodies (acetoacetate and beta hydroxybutyrate) that enter the TCA cycle in the mitochondria for generation of ATP.

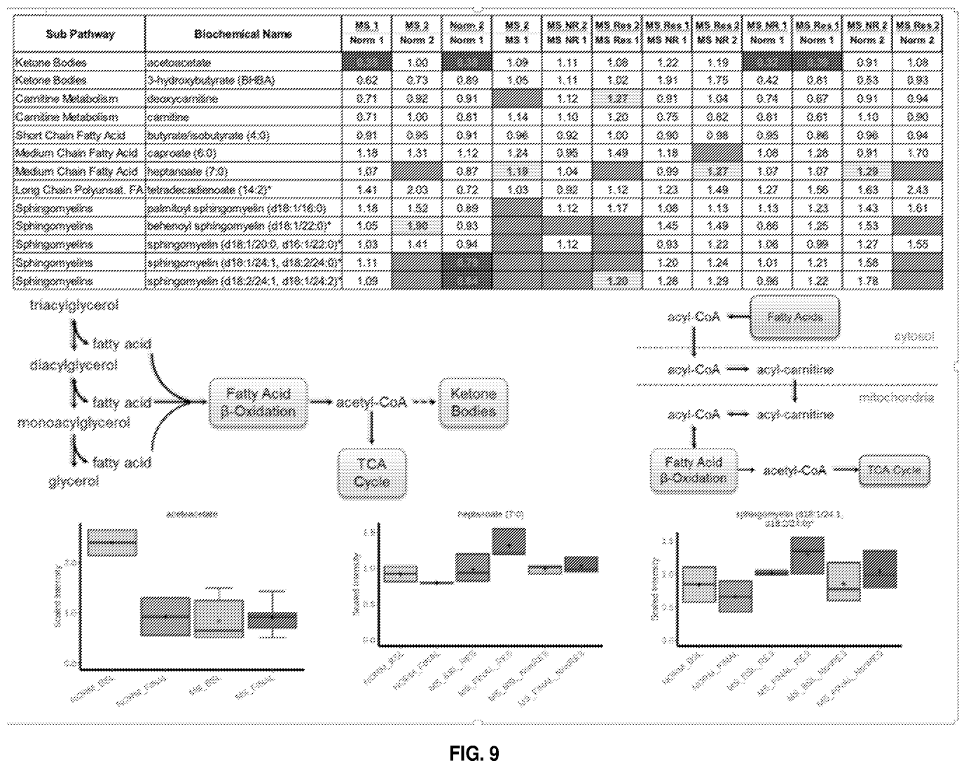

FIG. 10 shows normal mitochondria (green) are damaged (red) by oxidative stress and free radicles at sites of inflammation. When they accumulate in a neuron it undergoes apoptosis.

### DETAILED DESCRIPTION

This disclosure relates generally to methods for increasing brain histamine levels in multiple sclerosis (MS) patients. Also disclosed herein are compositions and methods for treating multiple sclerosis and fatigue associated with multiple sclerosis.

### 1. Definitions

Unless defined otherwise, all technical and scientific terms used herein have the meaning commonly understood by a person skilled in the art to which the claimed invention belongs. The terminology used herein is for describing particular embodiments only and is not intended to be limiting of the claimed invention. All technical and scientific terms used herein have the same meaning.

The following terms may have meanings ascribed to them below, unless specified otherwise. However, it should be understood that other meanings known or understood by those having ordinary skill in the art are also possible, and within the scope of the claimed invention. All publications, patent applications, patents, and other references mentioned or discussed herein are expressly incorporated by reference in their entireties. In the case of conflict, the present specification, including definitions, will control. In addition, the materials, methods, and examples are illustrative only and not intended to be limiting.

As used herein, the singular forms “a,” “and,” and “the” include plural references, unless the context clearly dictates otherwise or is otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Unless specifically stated or obvious from context, as used herein, the term “about” is understood as within a range of normal tolerance in the art, for example, within two standard deviations of the mean. About can be understood as within 20%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

“Administration” or “administering” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition (e.g., multiple sclerosis or a symptom thereof). The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., multiple sclerosis or a symptom thereof). The terms also encompass pharmaceutically acceptable, pharmacologically active derivatives of beneficial agents specifically mentioned herein, including, but not limited to, vectors, polynucleotides, polypeptides, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vectors, polynucleotides, polypeptides, salts, esters, amides, proagents, conjugates, active metabolites, isomers, fragments, analogs, etc.

The term “increased” or “increase” as used herein generally means an increase by a statically significant amount; for the avoidance of any doubt, “increased” means an increase of at least 5% as compared to a reference level, for example an increase of at least about 10%, at least about 20%, at least about 30%, at least about 40%, at least about 50%, at least about 60%, at least about 70%, at least about 80%, at least about 90% or up to and including a 100% increase or any increase between 5-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

As used herein, the term “or” means, and is used interchangeably with, the term “and/or,” unless context clearly indicates otherwise.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “reduced”, “reduce”, “reduction”, or “decrease” as used herein generally means a decrease by a statistically significant amount. However, for avoidance of doubt, “reduced” or “decreased” means a decrease by at least 5% as compared to a reference level, for example a decrease by at least about 10%, at least about 20%, at least about 30%, at least about 40%, at least about 50%, at least about 60%, at least about 70%, at least about 80%, at least about 90% or up to and including a 100% decrease (i.e. absent level as compared to a reference sample), or any decrease between 5-100% as compared to a reference level so long as the decrease is statistically significant.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

As used herein, the term “such as” means, and is used interchangeably with, the phrase “such as, for example” or “such as but not limited.”

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. Prophylactic treatments are administered to a subject prior to onset (e.g., before obvious signs of multiple sclerosis), during early onset (e.g., upon initial signs and symptoms of multiple sclerosis), or after an established development of multiple sclerosis. Prophylactic administration can occur for several days to years prior to the manifestation of symptoms of an infection.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of multiple sclerosis or a symptom thereof. In some embodiments, a desired therapeutic result is the alleviation of fatigue. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

### 2. Multiple Sclerosis and Histamine

Multiple Sclerosis (MS) is an autoimmune disease in which the immune system degrades the myeline sheath found on nerve cells. The disease is characterized as a brain and spinal cord (central nervous system) conditions, and leads to interference in nerve signal transmission. Signs and symptoms of MS vary widely however fatigue is common in MS patients.

The mechanism of fatigue associated with multiple sclerosis (MS) is unknown. Experienced by over 75% of patients with MS at some time during the course of their disorder, this severely disabling symptom is the cause of unemployment in a large number of patients with MS. Further, this symptom interferes with and worsens other symptom of MS especially depression, cognition, pain and motor function amongst others. Treatment of fatigue may alleviate these other symptoms of MS to the extent that the symptoms are worsened by fatigue, but each of these symptoms have their own mechanisms of pathogenesis. Accordingly, while patients may report improvement of cognition when their fatigue is successfully treated, cognitive dysfunction in MS cannot be treated by simply, alleviation of fatigue. Alleviating fatigue will have a significant impact on the overall quality of life of the individual.

Current agents that alleviate fatigue in MS are generally stimulants, and they alleviate fatigue through enhancement of the function of the noradrenergic brain. Stimulants of the phenethylamine class, dominated by the amphetamine salts, both levo and dextro isomers, used for treatment of attention deficit and sleep disorders have also found use in treatment of fatigue in MS. Ritalin (methylphenidate), Adderall and Adderall XR (racemic mixture of levo and dextro amphetamine salts) are examples of such drugs. Amantadine, a dopaminergic drug, and hence its use in Parkinsonism, probably work by increasing CNS dopamine, a step removed from nor-epinephrine. Modafinil, the most used agent for fatigue in MS acts through mechanisms that are yet to be defined. Modafinil stimulates discreet areas of the hypothalamus, but none that are directly nor adrenergic in its function. The orexin-hypocretin systems in the hypothalamus is activated, and indirectly though connections, the tuberomammillary system.

The tuberomammillary system is one of the oldest parts of the brain, and projects to every part of the neocortex and cerebellum and spinal cord and activate a variety of neurotransmitters. The tuberomammillary nucleus is the brain repository for histaminergic neurons similar to the locus coeruleus for nor epinephrine, or the nucleus basalis of Meinert is for acetylcholine. There are approximately 67,000 histaminergic neurons on each side.

Located in the posterior hypothalamus, its neurons project to the cerebral cortex, the hippocampus, neostriatum, nucleus accumbens, amygdala, cerebellum and spinal cord, and receive projections from the biological master clock, the suprachiasmatic nucleus. It is integrally associated with the circadian rhythm, has connections to the orexin-hypocretin system and is involved with arousal, sleep-wake cycles, learning and memory, satiety and hunger, libido and sexual arousal, and energy balance. Accordingly, increasing brain histamine can increase a variety of neurotransmitters involved in a multitude of brain functions especially arousal, energy, learning and memory.

Integrally associated with the suprachiasmatic nucleus, which is the master clock, this system is responsible for control of the circadian rhythm, wakefulness and sleep, satiety and hunger, libido and sexual arousal, and learning and memory. The sole neurotransmitter of these neurons is histamine. Blocking brain histamine receptors lead to tiredness and sleep.

Three types of receptors are identified for histamine within the brain. The H1 receptors are expressed on the post synaptic non histaminergic neurons in the cerebral cortex to which the histaminergic neurons project and these are involved with vigilance, attention and feeding. Increased vigilance and attention are associated with decreased feeding. The H2 receptors are seen on the post synaptic terminals of non-histaminergic cholinergic neurons in the hippocampus and are involved with increased learning and working memory mediated through release of acetylcholine. The H3 receptors are autocrine receptors that shut-off histamine release and are located presynaptically on the histaminergic neurons. Release of histamine into the synaptic cleft activate these receptors presynaptically on the histaminergic neurons and shut-off histamine thereby regulating its release.

Systemic administration of histamine cannot change the brain levels of histamine, since histamine is denied access to the central nervous system (CNS) across the blood-brain barrier. Accordingly, the disclosure herein increases brain histamine by administration of the histamine precursor L-histidine, an amino acid that has free access to the CNS through the amino acid receptors.

Accordingly, in some aspects, disclosed herein is a method for treating multiple sclerosis in a subject in need thereof, comprising administering to the subject a therapeutically effective amount of L-histidine.

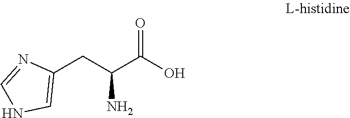 [SMILES: N[C@@H](Cc1c[nH]cn1)C(=O)O]

In some embodiments, L-histidine is administered orally, intravenously, and/or intramuscularly. In some embodiments, L-histidine is administered orally. In some embodiments, L-histidine is administered intravenously. In some embodiments, L-histidine is administered intramuscularly.

In order to alleviate side effects associated with peripheral conversion of L-histidine to histamine, as disclosed herein, conversion of L-histidine to histamine peripherally was blocked with L-aromatic amino acid decarboxylase inhibitor, carbidopa. Carbidopa is unable to access the CNS and therefore does not interfere with the central conversion of L-histidine to histamine. One of skill in the art will understand that histidine decarboxylase is responsible for catalyzing the decarboxylation of histidine to form histamine.

Accordingly, in some embodiments, the method disclosed herein further comprises administering to the subject a therapeutically effective amount of carbidopa.

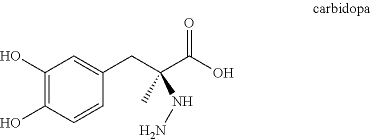 [SMILES: C[C@@](Cc1ccc(O)c(O)c1)(NN)C(=O)O]

Dosing frequency for the composition disclosed herein, includes, but is not limited to, at least once every month, once every three weeks, once every two weeks, once a week, twice a week, three times a week, four times a week, five times a week, six times a week, or daily, twice a day, three times a day, four times a day, or five times a day. In some embodiments, the interval between each administration is less than about less than about a month, less than about 3 weeks, less than about 2 weeks, or less than less than about a week, such as less than about any of 6, 5, 4, 3, 2, or 1 day. In some embodiments, the dosing frequency for the composition includes, but is not limited to, at least once a day, twice a day, or three times a day. In some embodiments, the interval between each administration is less than about 48 hours, 36 hours, 24 hours, 22 hours, 20 hours, 18 hours, 16 hours, 14 hours, 12 hours, 10 hours, 9 hours, 8 hours, or 7 hours. In some embodiment, the interval between each administration is less than about 24 hours, 22 hours, 20 hours, 18 hours, 16 hours, 14 hours, 12 hours, 10 hours, 9 hours, 8 hours, 7 hours, or 6 hours. In some embodiment, the interval between each administration is constant. For example, the administration can be carried out daily, every two days, every three days, every four days, every five days, or weekly. Administration can also be continuous and adjusted to maintaining a level of the compound within any desired and specified range.

In some embodiments, the carbidopa and L-histidine are taken daily. One of skill in the art will understand alternate dosing mechanisms can be utilized.

The dose of carbidopa can range from about 1 mg per day to about 1000 mg per day. In some embodiments, about 75 mg of carbidopa is used to maximally inhibit all histidine decarboxylase activity. The dose of carbidopa applied herein can be about 1 mg per day, about 5 mg per day, about 10 mg per day, about 15 mg per day, about 20 mg per day, about 25 mg per day, about 30 mg per day, about 35 mg per day, about 40 mg per day, about 45 mg per day, about 50 mg per day, about 55 mg per day, about 60 mg per day, about 65 mg per day, about 70 mg per day, about 75 mg per day, about 80 mg per day, about 85 mg per day, about 90 mg per day, about 95 mg per day, about 100 mg per day, about 120 mg per day, about 140 mg per day, about 160 mg per day, about 180 mg per day, about 200 mg per day, about 240 mg per day, about 280 mg per day, about 300 mg per day, about 350 mg per day, about 400 mg per day, about 450 mg per day, about 500 mg per day, about 550 mg per day, about 600 mg per day, about 650 mg per day, about 700 mg per day, about 750 mg per day, about 800 mg per day, about 850 mg per day, about 900 mg per day, about 950 mg per day, or about 1000 mg per day. In some embodiments, the dose of carbidopa is from about 25 mg per day to about 75 mg per day. In some embodiments, the dose of carbidopa is about 50 mg per day.

The dose of L-histidine applied herein can range from about 1 mg per day to about 10,000 mg per day. In some embodiments, the L-histidine dose is about 250 mg to 2000 mg per day. In some embodiments, the dose of L-histidine administered to a subject is from about 250 mg per day to about 1000 mg per day. In one embodiment the dose is 250 mg of L-histidine per day. In some embodiments, the dose is 500 mg of L-histidine per day. In some embodiments, the dose of L-histidine is 1000 mg per day. The daily dose of L-histidine can be about 250 mg per day, about 275 mg per day, about 300 mg per day, about 325 mg per day, about 350 mg per day, about 375 mg per day, about 400 mg per day, about 425 mg per day, about 450 mg per day, about 475 mg per day, about 500 mg per day, about 525 mg per day, about 550 mg per day, about 575 mg per day, about 600 mg per day, about 625 mg per day, about 650 mg per day, about 675 mg per day, about 700 mg per day, about 725 mg per day, about 750 mg per day, about 775 mg per day, about 800 mg per day, about 825 mg per day, about 850 mg per day, about 875 mg per day, about 900 mg per day, about 925 mg per day, about 950 mg per day, about 975 mg per day, about 1000 mg per day, about 1025 mg per day, about 1050 mg per day, about 1075 mg per day, about 1100 mg per day, about 1125 mg per day, about 1150 mg per day, about 1175 mg per day, about 1200 mg per day, about 1225 mg per day, about 1250 mg per day, about 1275 mg per day, about 1300 mg per day, about 1325 mg per day, about 1350 mg per day, about 1375 mg per day, about 1400 mg per day, about 1425 mg per day, about 1450 mg per day, about 1475 mg per day, about 1500 mg per day, about 1525 mg per day, about 1550 mg per day, about 1575 mg per day, about 1600 mg per day, about 1625 mg per day, about 1650 mg per day, about 1675 mg per day, about 1700 mg per day, about 1725 mg per day, about 1750 mg per day, about 1775 mg per day, about 1800 mg per day, about 1825 mg per day, about 1850 mg per day, about 1875 mg per day, about 1900 mg per day, about 1925 mg per day, about 1950 mg per day, about 1975 mg per day, or about 2000 mg per day.

The dose of L-histidine applied herein can be about 1 mg, about 10 mg, about 20 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1125 mg, about 1150 mg, about 1175 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, or about 2000 mg.

In some embodiments, the dose of carbidopa is unchanged. In some embodiments, the dose of carbidopa is increased or decreased over time. In some embodiments, the dose of L-histidine is unchanged. In some embodiments, the dose of L-histidine is increased or decreased over time. In some examples, the dose of carbidopa is unchanged and the dose of L-histidine is increased over a certain period of time (for example, over a one week period, a two week period, a three week period, a four week period, a five week period, a six week period, a seven period, an eight week period, a two month period, a three month period, a four month period, a five month period, a six month period, a seven month period, an eight month period, a nine month period, a ten month period, a eleven month period, a one year period, a five year period, or a ten year period). In some embodiments, the therapeutically effective amount of carbidopa is unchanged and the therapeutically effective amount of L-histidine is increased over a three week period.

The conversion of L-histidine to histamine outside the central nervous system is blocked by carbidopa which has no access to the central nervous system. Thereby, the combination of L-histidine and carbidopa allows for smaller doses of L-histidine to have a more robust effect since its degradation in the body is blocked by carbidopa, but such conversion to histamine in the central nervous system is not. This dose finding study has identified a dose of carbidopa+L-histidine that treats multiple sclerosis or symptoms thereof or alleviates fatigue without untoward side effects.

Carbidopa can be administered concurrently, before, or after L-histidine. In some embodiments, carbidopa and L-histidine are contained within one composition. Accordingly, in some aspects, disclosed herein is a composition comprising carbidopa and L-histidine. In some embodiments, the composition further comprises a pharmaceutically acceptable carrier.

In a further embodiment is a method for alleviating fatigue in individuals with multiple sclerosis, the method comprising oral administration of L-histidine.

Multiple sclerosis can result in numbness or weakness in one or more limbs, electric-shock sensations that occur with certain neck movements, tremor, lack of coordination or unsteady gait, vision problems (partial or complete loss of vision, blurry vision, or prolonged double vision), slurred speech, fatigue, dizziness, tingling or pain in parts of body, and/or problems with sexual, bowel, or bladder function. Accordingly, it should be understood that a treatment of multiple sclerosis can be a treatment of one or more of numbness or weakness in one or more limbs, electric-shock sensations that occur with certain neck movements, tremor, lack of coordination or unsteady gait, vision problems (partial or complete loss of vision, blurry vision, or prolonged double vision), slurred speech, fatigue, dizziness, tingling or pain in parts of body, and/or problems with sexual, bowel, or bladder function. Multiple sclerosis can be diagnosed by MRI scan to detect lesions (areas of damage) in the brain or spinal cord that indicate multiple sclerosis or by spinal tap (lumbar puncture) that shows abnormality in antibodies that are associated with multiple sclerosis. Multiple sclerosis can also be diagnosed by the McDonald criteria. In some embodiments, the methods and compositions disclosed herein alleviate fatigue in the subject with multiple sclerosis.

Also disclosed herein is a method for treating, preventing, mitigating, and/or alleviating fatigue in a subject in need thereof, the method comprising administering to the subject a therapeutically effective amount of L-histidine. In some embodiments, the therapeutically effective amount of L-histidine is from about 250 mg per day to about 2000 mg per day. In some embodiments, the therapeutically effective amount of L-histidine is from about 250 mg per day to about 1000 mg per day. In some embodiments, L-histidine is administered orally, intravenously, or intramuscularly.

The severity of fatigue can be determined by Fatigue Severity Score (FSS) of 5.0 or greater (maximum mean FSS=7.0). Accordingly, a treatment or alleviation of fatigue can be indicated as decreased Fatigue Severity Score (FSS).

In some embodiments, the method disclosed herein further comprises administering to the subject a therapeutically effective amount of carbidopa. In some embodiments, the therapeutically effective amount of carbidopais from about 25 mg per day to about 75 mg per day. In some embodiments, the therapeutically effective amount of carbidopais about 50 mg per day. In some embodiments, carbidopa is administered concurrently, before, or after L-histidine. In some embodiments, carbidopa and L-histidine are administered orally.

In some embodiments, the therapeutically effective amount of carbidopais unchanged and the therapeutically effective amount of L-histidine is increased over time (e.g., over a three week period).

In some embodiments, the subject has multiple sclerosis.

Accordingly, in some aspects, disclosed herein is a method for alleviating fatigue in individuals with MS, the method comprising administering a therapeutically effective amount of carbidopa and L-histidine.

One of skill in the art will understand that an example of one brand name for the generic drug carbidopa is lodosyn.

In yet another embodiment is a method for alleviating fatigue in individuals, the method comprising administering a therapeutically effective amount of carbidopa and L-histidine.

Also, it should be understood and herein contemplated that the methods and/or compositions provided herein can improve mitochondrial function of neural system. In some embodiments, the methods and/or compositions provided herein increase a level of N-acetyl aspartate in the central nervous system of the subject who receive the treatment. In some embodiments, the methods and/or compositions provided herein increase a level of creatine phosphate in the central nervous system of the subject who receive the treatment. In some embodiments, the methods and/or compositions provided herein increase a level of deoxycarnitine in the central nervous system of the subject who receives the treatment.

The dosage forms can be adapted for administration by any appropriate route. Appropriate routes include, but are not limited to, oral (including buccal or sublingual), rectal, epidural, intracranial, intraocular, inhaled, intranasal, topical (including buccal, sublingual, or transdermal), vaginal, intraurethral, parenteral, intracranial, subcutaneous, intramuscular, intravenous, intraperitoneal, intradermal, intraosseous, intracardiac, intraarticular, intracavernous, intrathecal, intravitreal, intracerebral, gingival, subgingival, intracerebroventricular, and intradermal. Such formulations may be prepared by any method known in the art. In some embodiments, the carbidopa and L-histidine are administered orally. However, the L-histidine can also be administered intramuscularly or intravenously.

It is understood and herein contemplated that the timing of multiple sclerosis or fatigue onset can often not be predicted. The disclosed methods of treating, preventing, alleviating, mitigating, and/or inhibiting multiple sclerosis or fatigue can be used prior to or following the onset of multiple sclerosis or fatigue. In one aspect, the disclosed methods can be employed 30, 29, 28, 27, 26, 25, 24, 23, 22, 21, 20, 19, 18, 17, 16, 15, 14, 13, 12, 11, 10, 9, 8, 7, 6, 5, 4, 3, 2 years, 12, 11, 10, 9, 8, 7, 6, 5, 4, 3, 2 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2, or 1 hour prior to onset of multiple sclerosis or fatigue; or 1, 2, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 days, 3, 4, 5, 6, 7, 8, 9, 10, 11, 12 months, 2, 3, 4, 5, 6, 7, 8, 9, 10, 11, 12, 15, 18, 24, 30, 36, 48, 60 or more years after onset of multiple sclerosis or fatigue.

In some aspects, disclosed herein are methods for increasing brain histamine by administering the precursor L-histidine which retains the ability to access the CNS by way of amino acid transporters. Also disclosed herein are methods that overcome the limitations of peripherally administered histamine.

In one aspect is a method for alleviating fatigue in individuals with multiple sclerosis, the method comprising administration of a therapeutically effective amount of carbidopa and L-histidine.

In another embodiment, carbidopa and L-histidine are taken daily and the dose of carbidopa is unchanged and the dose of L-histidine is increased over a three week period.

In another embodiment, the dose of carbidopa is from 25 mg to 75 mg per day, or more specifically, 50 mg per day. Similarly, the doses of L-histidine are from 250 mg to 2000 mg per day, more specifically 250 mg to 1000 mg.

In another embodiment, the carbidopa is administered concurrently, before, or after the L-histidine and both are administered orally. In another aspect the L-histidine is administered intravenous (IV) and intramuscular (IM)

In an alternate embodiment is a method for alleviating fatigue in individuals with multiple sclerosis, the method comprising oral administration of L-histidine.

In yet another embodiment is a method for alleviating fatigue in individuals, the method comprising administration of a therapeutically effective amount of carbidopa and L-histidine.

As seen in the examples below, L-histidine has neuroprotective properties as shown by the improved mitochondrial function in neurons. The improvement of CNS mitochondrial function can also be used to treat other neurodegenerative diseases such as Alzheimer's Disease, Parkinson's disease, or Amyotrophic Lateral Sclerosis (ALS). In some aspects, disclosed herein is a method of a treating a neurodegenerative disorder in a subject in need thereof comprising administering a therapeutically effective amount of L-histidine. In other aspects, disclosed herein is a method of a treating a neurodegenerative disorder in a subject in need thereof comprising administering a therapeutically effective amount of L-histidine and/or carbidopa. See, for example, Han J, Park H, Maharana C, et al. Alzheimer's disease-causing presenilin-1 mutations have deleterious effects on mitochondrial function. Theranostics. 2021;11(18):8855-8873; Kumar R, Harilal S, Parambi DGT, et al. The Role of Mitochondrial Genes in Neurodegenerative Disorders. Curr Neuropharmacol. 2021; Xu S, Zhang X, Liu C, et al. Role of Mitochondria in Neurodegenerative Diseases: From an Epigenetic Perspective. Front Cell Dev Biol. 2021;9:688789; Barcelos I P, Troxell R M, Graves J S. Mitochondrial Dysfunction and Multiple Sclerosis. Biology (Basel). 2019;8(2):37. doi: 10.3390/biology8020037; Macdonald R, Barnes K, Hastings C, Mortiboys H. Mitochondrial abnormalities in Parkinson's disease and Alzheimer's disease: can mitochondria be targeted therapeutically? Biochem Soc Trans. 2018;46(4):891-909.

In some aspects, disclosed herein is a method of improving cognition in a subject comprising administering a therapeutically effective amount of L-histidine. In other aspects, disclosed herein is a method of improving cognition in a subject comprising administering a therapeutically effective amount of L-histidine and/or carbidopa.

### EXAMPLES

The following examples are set forth below to illustrate the compositions, methods, and results according to the disclosed subject matter. These examples are not intended to be inclusive of all aspects of the subject matter disclosed herein, but rather to illustrate representative compositions, methods, and results. These examples are not intended to exclude equivalents and variations of the present invention which are apparent to one skilled in the art.

### Example 1. L-Histidine Mitigates Multiple Sclerosis-Related Fatigue

### Groups

Three groups of subjects participated in a sequential dose escalation protocol. All subjects received a fixed dose of carbidopa, 50 mg po bid. One of skill in the art will understand that a minimum of 75 mg of carbidopa a day is necessary to maximally inhibit all histidine decarboxylase activity. Tablets of the study drugs were produced by the research pharmacy at the University of Miami. Carbidopa was prepared in 50 mg, and 1-histidine at 250 mg strength. Drug dispensation was carried out by the research pharmacist according to the dosage designated for each group.

Group 1 received 250 mg po bid of L-histidine in 250 mg strength tablets. Benefits and adverse events were documented and reported to the Data Safety Monitoring Body (DSMB). After approval from the DSMB, group 2 was started. Group 2 received 500 mg po bid of L-histidine. Once again after approval from the DSMB, following the successful completion of group 2, escalation to group 3, namely 1 gm po bid of L-histidine was completed. All subjects agreed to participate with 2 lumbar punctures each a week apart, for collection of cerebrospinal fluid (CSF) for the PK/PD part of the study. Each group required the participation of 3 MS subjects and 1 normal individual. For whatever reason if a subject could not complete the study, the design permitted substitution so every group will have three MS subjects and a normal subject that completed the trial.

After informed consent, subjects were screened at the first visit to meet inclusion/exclusion criteria and baseline laboratory studies were obtained including complete blood counts, comprehensive metabolic panel, thyroid panel, and inventory of health measures, including a complete general examination as well as EKG. Subjects completed questionnaires that included the following: Visual Analogue Fatigue Scale (VAFS), fatigue severity scale (FSS), modified fatigue impact scale (MFIS), Epworth sleep scale, Satiety scale, and Center for Epidemiologic Studies Depression Scale. Within 30 days of their screening visit, subjects returned for their baseline visit. The questionnaires were completed again, and subjects underwent the baseline lumbar puncture for collection of baseline cerebrospinal fluid (CSF).

The study medication for the appropriate group was dispensed and all subjects took the first dose of the study drug during this visit. Subjects were observed for an hour for side effects if any and instructed on how to take the medication on a BID regimen.

Medications were dispensed for 7 days. On day 8, subjects arrived at the center and received the final dose of the study drug and timed from that dosing for the PK/PD studies.

Blood samples were collected at drug administration, and every 30 minutes for 4 hours. At 90 minutes the second and final lumbar puncture was completed, and CSF obtained for measurement of L-histidine and histamine. All subjects completed the same questions to determine the effects of the treatment on fatigue, wakefulness and satiety.

### Subjects

All subjects were recruited from the MS Center at the University of Miami. Subjects who complained of severe and persistent fatigue of duration longer than 6 months were eligible to be screened for the study. During screening inclusion and exclusion criteria were carefully reviewed. To be eligible, a subject needed to have a definite diagnosis of MS by the McDonald International Criteria and severe fatigue with a mean Fatigue Severity Score (FSS) of 5.0 or greater (maximum mean FSS=7.0). Patients with confounding factors such as anemia, untreated hypothyroidism, depression, concomitant medication that caused fatigue were carefully examined and excluded. Use of stimulants and concomitant use of medications for treatment of fatigue such as modafinil was excluded. Use of caffeine was not restricted, but patients were instructed not to change the usage. Pregnant or lactating females were not included in the study. Patients were cautioned not to use over-the-counter medications without approval of the PI during the study, especially medications for sleep or allergies including but not limited to the use of oral steroids or antihistamines with potential to confound the results of the trial.

Normal subjects were individuals who did not have MS, were healthy, and did not have complaints of fatigue. They were spouses of patients who participated in this trial.

### Responder Analysis

At the completion of the study visits each subject was designated as a responder or non-responder as defined herein based on the following criteria: A response to the treatment was based on the scores for fatigue scales on-drug vs. off-drug (the average of the scores at screening and baseline). To be considered a responder a subject had to respond better by improved scores on-drug vs off-drug in all 3 measures of fatigue scales namely the VAFS, FSS and show a 1 point improvement on-drug vs. off-drug in the FSS or a 20% improvement in the MFIS.

### Pharmacokinetic (PK) and Pharmacodynamic (PD) Studies

On day 7 from the baseline, the end-of-study visit, subjects arrived at the center without taking the last dose, which was administered under supervision and timed for time-zero. Baseline samples of blood for serum and plasma were collected and blood samples were obtained every half-hour for 6 hours.

At 90 minutes post administration a lumbar puncture was performed, and 0.5 ml aliquots of spinal fluid were collected for a total of 5 minutes. Spinal fluid, serum, and plasma were frozen at -80C until analysis for levels of histidine, histamine and metabolomic studies.

### L-Histidine Measurement

L-Histidine and L-Histidine-¹³G₆ analytical standards (Sigma Aldrich) were prepared in LC-MS grade water with 0.1% formic acid (FA). 25 μL of sample (CSF or blood) or calibration standard was mixed with 75 μL of LC-MS grade water with 0.1% FA and 200 μL of 2.5 μM L-Histidine-¹³G₆. The samples were vortexed for approximately 1 minute and centrifuged at 2,700 ×g for 15 minutes at 4° C. The supernatant was removed and 5 μL was injected onto an Acclaim™ Polar Advantage II C18 column (Thermo Scientific) using an Accela 600 HPLC (Thermo Scientific). Solvent A was water with 0.1% FA and Solvent B was acetonitrile with 0.1% FA. All solvents were LC-MS grade. The gradient started at 10% solvent B and was held for 0.5 minutes, switched to 80% solvent B over 1 minute and was held for 0.5 min. The system switched to starting conditions over 0.1 minute and re-equilibrated for 0.8 minutes. The column temperature was 30° C. and the flow rate was 350 μL/minute. L-Histidine and L-Histidine-¹³G₆ were ionized using a heated electrospray ionization source (HESI) with a sheath gas and auxiliary gas flow rate of 40 and 10 arbitrary units, respectively. The spray voltage was set to 4 kV, the capillary temperature was 325° C. and the S-lens RF level was 50. The data was acquiring using a Q-Exactive Orbitrap mass spectrometer (Thermo Scientific) using a parallel reaction monitoring (PRM) experiment in positive mode. The mass spectrometer was operated using a resolution of 140,000, AGC target of 1e6, maximum injection time of 400 ms, loop count of 2, isolation window of 0.4 m/z and normalized collision energy of 30. A calibration curve was created using X Calibur (Version 4.1) Quan browser software. The peak areas were normalized against those of the internal standard and plotted to calculate absolute concentrations.

### Responder Analysis

None of the normal subjects reported any fatigue and did not notice any benefit from the study drug. Nor did they experience any adverse events and did not report any overall added benefit.

### Group 1:

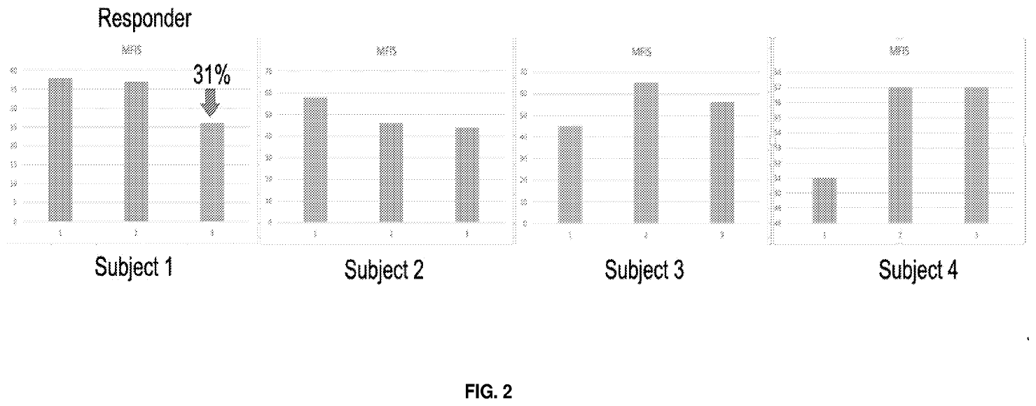

One individual in Group 1 (150 mg po bid of L-histidine and carbidopa 50 mg po bid) had the surprising technical effect of improved fatigue scores in all three fatigue scales and a 30% improvement in the MFIS (FIG. 2). No adverse events were reported.

### Group 2:

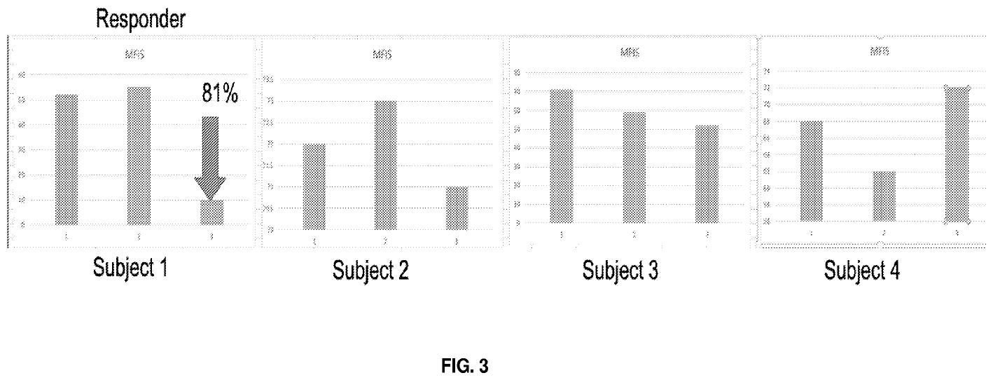

At a dose of 500 mg po bid of L-histidine and carbidopa 50 mg po bid, a single subject noted improvement in all 3 fatigue scales, and the response to treatment was described as “remarkable and outstanding”. This subject recorded an 81% improvement in the MFIS (FIG. 3)

In a written narrative the subject reported the unexpected technical effects of improvement beyond what he had previously experienced with any agent including modafinil. The subject reported being able to think clearly, have better focus, and improved cognition.

Remarkable improvement of libido and sexual activity was reported in the written narrative, and a marked improvement in overall quality of life.

### Group 3:

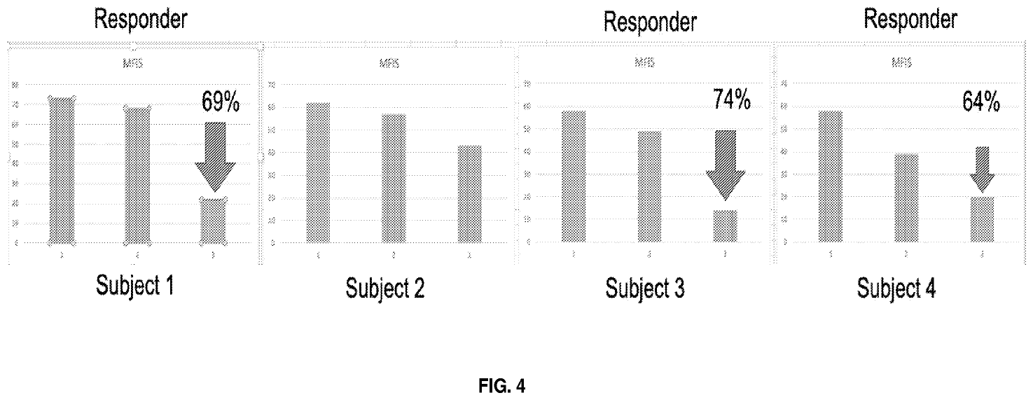

At L-histidine doses of 1 gm po bid and carbidopa 50 mg po bid, three of the three MS subjects noticed remarkable improvement. All subjects fulfilled all criteria for response and improvements of MFIS at 69%, 74% and 82% better than off-drug scores (FIG. 4). In their written narratives these subjects also reported that the “brain fog” lifted, and they could focus better and think clearly. The relief from fatigue was complete. Two additional subjects enrolled in this group withdrew, for adverse side effects considered unrelated to the study medications and both subjects withdrew from the study.

In all subjects who noted improvement, at whatever dose, subjects noticed the change usually on day 2, and the response was sustained until the study drug was discontinued. All responders expressed their desire to continue the study drug.

### Response to Sleep

Most subjects in this study reported normal sleep scores. Epworth Sleep Scales (ESS) were over 10 in only 3 subjects and one of these subjects responded to the study drug with marked improvement of the fatigue. A significant drop in the ESS was observed in this responder subject whose ESS dropped from 13 to 6, a significant improvement.

### Satiety Scales

A change in satiety scale was not recorded in this study where the exposure to the study drug was short. A change in weight was also not recorded.

### Adverse Events

Reported adverse events were few and related to the lumbar punctures or unrelated intercurrent infections. One normal subject experienced a post-lumbar puncture headache after the completion of the study after the second and final lumbar puncture, that resolved after conservative measures without a blood patch. A second subject with MS experienced post lumbar puncture headaches at baseline and chose not to continue the study. Another MS subject in group 2 who had significant difficulties with memory returned most of the study medications as he forgot to take the study drugs as prescribed and did not complete the study. As mentioned above, two subjects had to withdraw from infectious complications determined as unrelated to the study. One patient had a Bartholin's abscess that required incision and drainage and treatment with antibiotics and the second subject had what was suspected to be COVID-19 infection and also underwent treatment with antibiotics and withdrew.

### PK and PD Studies

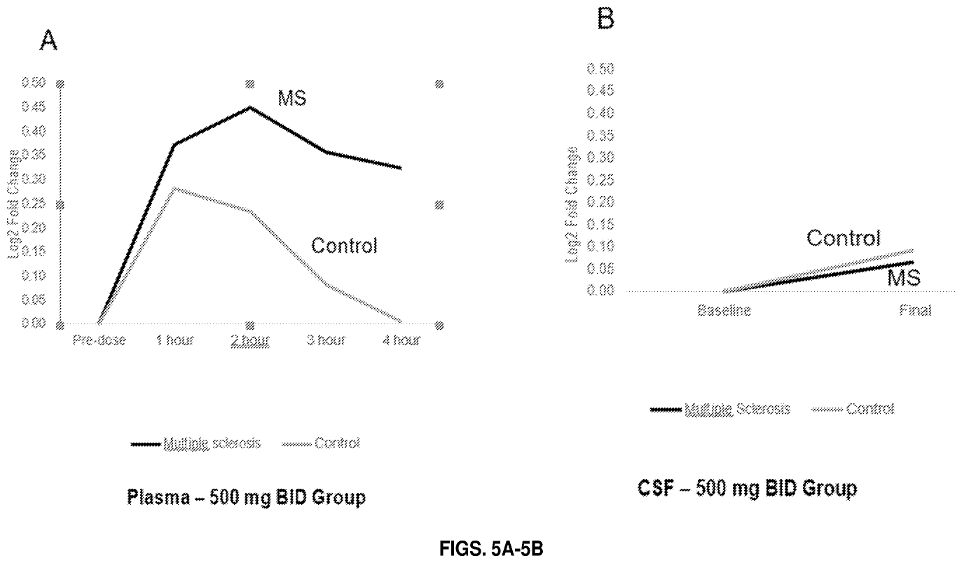

As shown in FIG. 5, peak levels of L-histidine were recorded at 2 hours in the plasma and the levels gradually declined and eventually became undetectable after 4 hours. The increased levels of L-histidine at 90 minutes in the spinal fluid clearly showed the effect of I-histidine loading as a successful intervention in increasing the levels of the amino acid in the central nervous system.

As disclosed herein, a dose-response pattern was observed with a modest response of improvement in fatigue at the lowest dose in a single subject, a robust response in a single subject at the intermediate dose and a robust response in all subjects at the highest dose. The magnitude of the responses reported appear clinically meaningful with subjects experiencing improved quality of life. These early observations provide surprising technical effects lend itself to encouragement to examine this approach for treatment of fatigue in patients with MS who experience this symptom. The approach is novel and open a whole new way to treat fatigue.

Moreover, most responders of fatigue also reported improved attention, concentration and improvement of “brain fog” indicating that improvement in cognition is another outcome. A post-hoc examination of the subset scores of MSFC identified improvement of all three domains, physical, cognitive and psychosocial. Further, while not all subjects with fatigue had elevated Epworth Sleep Scales (ESS), only 3 subjects had sleep scores above 10 and only one of these was a fatigue responder. In that single individual, a remarkable improvement of the ESS was also noted concomitant with the improvement of all fatigue scores.

The tuberomammillary nucleus is the brain repository for histaminergic neurons similar to the locus coeruleus for nor epinephrine, or the nucleus basalis of Meinert is for acetylcholine. There are approximately 67,000 histaminergic neurons on each side. Located in the posterior hypothalamus, its neurons project to the cerebral cortex, the hippocampus, neostriatum, nucleus accumbens, amygdala, cerebellum and spinal cord, and receive projections from the biological master clock, the suprachiasmatic nucleus. It is integrally associated with the circadian rhythm, has connections to the orexin-hypocretin system and is involved with arousal, sleep-wake cycles, learning and memory, satiety and hunger, libido and sexual arousal, and energy balance. Accordingly, increasing brain histamine can increase a variety of neurotransmitters involved in a multitude of brain functions especially arousal, energy, learning and memory.

Three types of receptors are identified for histamine within the brain. The H1 receptors are expressed on the post synaptic non histaminergic neurons in the cerebral cortex to which the histaminergic neurons project and these are involved with vigilance, attention and feeding. Increased vigilance and attention are associated with decreased feeding. The H2 receptors are seen on the post synaptic terminals of non-histaminergic cholinergic neurons in the hippocampus and are involved with increased learning and working memory mediated through release of acetylcholine. The H3 receptors are autocrine receptors that shut-off histamine release and are located presynaptically on the histaminergic neurons. Release of histamine into the synaptic cleft activate these receptors presynaptically on the histaminergic neurons and shut-off histamine thereby regulating its release.

This application refers to various journal articles, and other publications, which are incorporated herein by reference. If there is a conflict between any of the incorporated references and the instant specification, the specification shall control. In addition, any particular embodiment of the present invention that falls within the prior art can be explicitly excluded from any one or more of the claims. Because such embodiments are deemed to be known to one of ordinary skill in the art, they can be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

### Example 2. Metabolomics in the Cerebrospinal Fluid After Histidine Loading

### Methods

Four CSF samples from normal subjects (2 before histidine loading and 2 after) and 12 CSF samples from MS patients (6 before and 6 after histidine loading) were examined in this study. The MS samples were evenly distributed from 3 responders and 3 non-responder MS subjects.

The dataset comprised a total of 494 biochemicals, 434 known and 60 unknown entities. Following log transformation and imputation of missing values if any with the minimum observed value for each compound, ANOVA contrasts were used to identify biochemicals that differed significantly between experimental groups. A summary of the biochemicals that achieved significance (p≤0.05) as also those approaching significance (0.05<p<0.10), were examined. All assays also examined estimates of false discoveries (q-value) inherent in estimates that use multiple comparisons. A low q-value (<0.1) gave confidence in a result with a low p value as a potentially meaningful result.

Glycolytic end products pyruvate and lactate are increased/trending higher in MS subjects compared to normal, at both time points. This indicates increased glycolysis in the CSF of subjects with MS. As pyruvate can enter the TCA cycle forming oxaloacetate, or via acetyl-CoA, forming citrate, the increases/higher trends observed in citrate and cis-aconitate in the post dose samples from MS subjects indicates higher TCA cycle activity due to histidine loading. Isocitrate is decreased/trending lower in the CSF of MS subjects that positively responded to histidine loading when compared to non-responders and normal subjects, at both time points, indicating lower cerebral TCA cycle activity.

Several lines of evidence identify improved mitochondrial function after histidine loading in MS subjects regardless of their responder status. First, pyruvate and lactate were increased in MS patients compared to normal subjects, because of increased glycolysis. Post dosing, increases were observed for citrate and cis-aconitate secondary to improved engagement of the TCA cycle and improved generation of ATP, which was also reflected in the increases in phosphocreatine observed after histidine loading. Decreased creatine phosphate in the pre-dose CSF of MS subjects compared to normal subjects, indicating a perturbation in energy homeostasis in subjects with multiple sclerosis was observed. This lower creatine phosphate appears to be mitigated to an extent by histidine loading, more so in MS responders compared to MS non-responders.

Deoxycarnitine, the precursor of carnitine was increased after histidine loading. Carnitine is important for the transport of long-chained fatty acids across mitochondrial membrane, where it is metabolized by beta oxidation to ketone bodies. Acetoacetate and beta hydroxybutyrate which were lower prior to loading, increased following histidine loading, probably because of improved metabolism of long chain fatty acids. The ketone bodies are neuroprotective and also the source of efficient energy production since they are metabolized through the mitochondrial TCA cycle. Collectively, improved mitochondrial function leads to improved energy states and improved overall neuronal function.

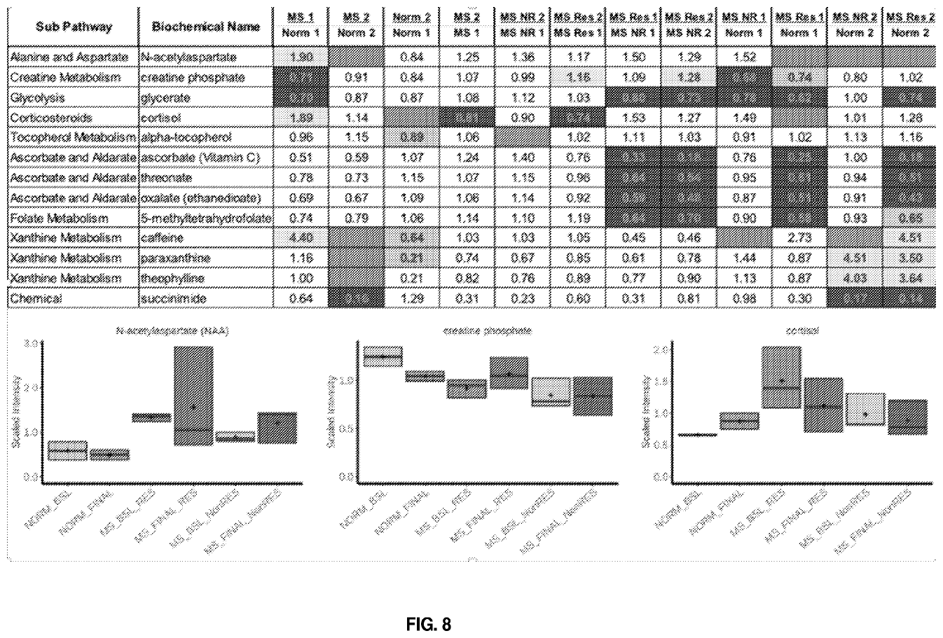

N-acetyl aspartate (NAA) is used in magnetic resonance spectroscopy (MRS) as a marker of neuronal health. NAA is exclusively produced by mitochondria in neurons and its measurement by MRS in white matter of the brain is used as a surrogate for axonal health in patients with MS. Few studies have examined the usefulness of measurement of NAA in the CSF in MS. When examined in MS, a lower level of CSF NAA correlated to increasing disease severity and disability. By contrast, elevated NAA levels in the first 4 days in the CSF was a predictor of poor outcomes after traumatic brain injury. In this study, NAA levels were increased after histidine loading, probably reflecting improved mitochondrial function in neurons (FIG. 8).

Of note, trends in microbiome-associated products of histidine: imidazole lactate and 4-imidazoleacetate were observed in the CSF samples, which reflected changes associated with the microbiome. Both imidazole lactate and 4-imidazoleacetate were trending lower in MS responder subjects compared to non-responder subjects at both time points. In view of the current interest in gut microbiome, the presence of these microbiome derived imidazole compounds in the CSF are of particular interest, the significance of which is yet uncertain.

The most significant findings from these studies are the identification that the energy state of the brain is improved after the intervention and multiple converging lines of evidence point to improved mitochondrial function as the basis for this. The baseline low levels of phosphocreatine observed in the CSF in this study are in keeping with previous observations of decreased glucose utilization by the MS brain described in FDG PET studies. As indicated earlier, the lower creatine phosphate was mitigated to an extent by L-histidine loading, more so in MS responders compared to MS non-responders (FIG. 8).

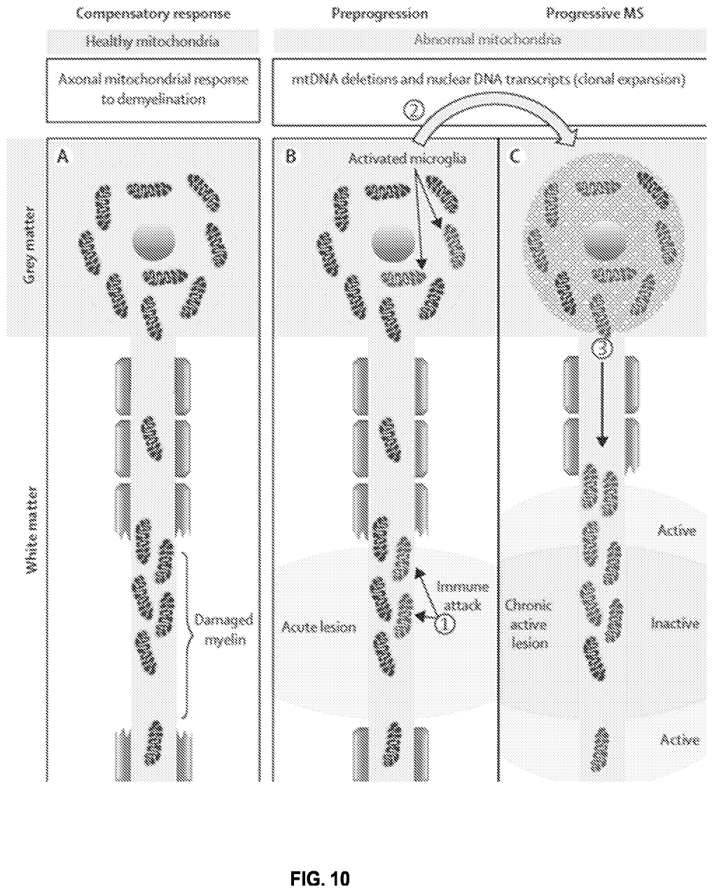

Improving mitochondrial function can improve bio energetics and improve neuronal health and survival. Early loss of neurons secondary to mitochondrial dysfunction has been implicated in the pathogenesis of progressive disease. As shown in the figure, accumulation of defective mitochondria ultimately leads to compromised energy state in the neuron resulting in apoptosis. The compositions and methods described herein can improve neuronal survival and delay development of progressive MS (FIG. 10). Additionally, the intervention also increases the levels of carnosine and homocarnosine, both known to have neuroprotective properties. Collectively, it shows that histidine loading can improve not only fatigue and cognition but also the underlying disease process to favorably affect the long-term brain degeneration in MS for which there are no effective therapies.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method for treating multiple sclerosis in a subject in need thereof, comprising administering to the subject a therapeutically effective amount of L-histidine.

2. The method of claim 1, wherein L-histidine is administered orally, intravenously, or intramuscularly.

3. The method of claim 1, further comprising administering to the subject a therapeutically effective amount of carbidopa.

4. The method of claim 3, wherein carbidopa and L-histidine are taken daily.

5. The method of claim 3, wherein the therapeutically effective amount of carbidopa is unchanged and the therapeutically effective amount of L-histidine is increased over a certain period of time.

6. The method of claim 5, wherein the therapeutically effective amount of L-histidine is increased over a three week period of time.

7. The method of any one of claims 3, wherein the therapeutically effective amount of carbidopais from about 25 mg per day to about 75 mg per day.

8. The method of claim 7, wherein the therapeutically effective amount of carbidopais about 50 mg per day.

9. The method of claim 3, wherein carbidopa is administered concurrently, before, or after L-histidine.

10. The method of any one of claim 3, wherein carbidopa and L-histidine are administered orally.

11. The method of any one of claim 1, wherein the therapeutically effective amount of L-histidine is from about 250 mg per day to about 2000 mg per day.

12. The method of claim 11, wherein the therapeutically effective amount of L-histidine is from about 250 mg per day to about 1000 mg per day.

13. The method of claim 1, wherein the method increases a level of N-acetylaspartate in the central nervous system of the subject.

14. The method of claim 1, wherein the method increases a level of creatine phosphate in the central nervous system of the subject.

15. The method of claim 1, wherein the method alleviates fatigue in the subject.

16. A method for alleviating fatigue in a subject in need thereof, the method comprising administering to the subject a therapeutically effective amount of L-histidine.

17.-29. (canceled)

30. The method of claim 16, wherein the subject has multiple sclerosis.

31. (canceled)

32. (canceled)

33. A method for treating a neurological disorder in a subject in need thereof, comprising administering to the subject a therapeutically effective amount of L-histidine.

34.-46. (canceled)

47. The method of claim 33, wherein the neurological disorder is Alzheimer's Disease, Parkinson's disease, or Amyotrophic Lateral Sclerosis (ALS)

48. The method of claim 33, wherein the method improves cognition in the subject.
